(19) United States
(12) Patent Application Publication
(10) Pub. No.: US 2009/0272204 A1
(43) Pub. Date: Nov. 5, 2009

(54) DEVICE AND PROCESS FOR REDUCING AN ETHANOL CONTENT OF A LIQUID PREPARATION

(75) Inventors: Michael Roloff, Oberweser-Arenborn (DE); Regina Peter, Brakel-Istrup (DE); Matthias Lutkenhaus, Uslar (DE); Katharina Reichelt, Rosenheim (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co., KG, Holzminden (DE)

(21) Appl. No.: 12/428,914

(22) Filed: Apr. 23, 2009

(30) Foreign Application Priority Data
Apr. 30, 2008 (EP) 08 155 462.8

(51) Int. Cl. G01N 33/00; B01D 3/14; A23P 1/00

(52) U.S. Cl. 73/865.7; 202/153; 426/489

(57) ABSTRACT

The present invention relates to a device and a process for reducing an ethanol content of a liquid preparation, to the use of the device for producing a liquid flavouring substance concentrate and to processes for sensory assessment thereof.

### CROSS-REFERENCE TO RELATED APPLICATION

This application claims benefit of priority to EP 08 155 462.8, filed on Apr. 30, 2008, which is incorporated herein by reference in its entirety.

### FIELD OF THE INVENTION

The present invention relates to a device and a process for reducing an ethanol content of a liquid preparation, to the use of the device for producing a liquid flavouring substance concentrate and to processes for sensory assessment thereof.

### PRIOR ART

Aromas are usually complex mixtures of a large number of sensorially active substances, such as for example plant extracts or natural, nature identical or synthetic aroma substances. Aroma compositions comprising or consisting of sensorially active substances contain at least two different aroma substances which can be volatile (odorous substances) or non-volatile (flavouring substances). In the human nose, volatile odorous substances can be perceived retronasally or orthonasally. The taste (gustatory) impressions umami, salty, sour, sweet and bitter are perceived by interaction of the flavouring substances with the taste receptors of the tongue; furthermore, it is possible to perceive in this case also further sensory impressions, for example prickling, cooling, sharp, burning or electrifying (“tingling”) effects which are often produced owing to trigeminal stimulations. The overall sensory impression of an aroma composition is fundamentally influenced by the quantitative ratios of the sensorially active substances of an aroma composition relative to one another, wherein what matters is the sensory contribution of the individual aroma substances and not the absolute quantity thereof. Sensorially relevant aroma substances contained in food products are frequently contained only in very low quantities and in many cases not yet known, although these aroma substances make, owing to their high sensory activity, a significant contribution to the odour and/or taste. An important assessment criterion is therefore the aroma value, defined as the quotient of the concentration of a sensorially active aroma substance relative to the odour or taste threshold value.

In the aroma industry, a large number of different sensory assessment processes or devices for sensory assessment are nowadays used. The samples used for assessment are in this case often prepared in a very labour and time-intensive manner. Highly volatile solvent components in sample extracts are frequently reduced using separate processes. Sample mixtures are produced by additional operations. For the individual applicant, it is extremely difficult, or almost impossible, to control the operation of the large number of different devices and processes. He is therefore necessarily restricted to the known and available devices and processes; this can lead to suitable devices or processes not being applied in specific cases. An article providing an overview of this topic has been published in the journal Chemie in unserer Zeit 2003, 37, 388-401, or the literature cited therein.

In the search for flavouring and odorous substances, there are to date no high throughput screening processes for sensory assessment of the test compounds known, which are used for rapid identification of new, sensorially interesting compounds. Therefore, the systematic examination of a natural substance extract nowadays presupposes multiple-stage separation and pretreatment to purify and concentrate the individual compounds contained in the extract. In particular, the preparation of samples for sensory assessment in different test media is very time-consuming in this regard. However, the formation of artifacts, instability or recovery losses during the isolation/blending of odorous and/or flavouring substances from/with complex matrices can also lead to odour or taste misinformation and continually presents the analyst with new challenges. A large number of different, gentle extraction and isolation processes, which in particular individually address specific objectives, have therefore been developed.

For the separation of aroma compositions by liquid chromatography, use is conventionally made of eluants (mobile phase) which are miscible with water, but present a risk to health or are poisonous. Frequently used eluants are acetonitrile, methanol and tetrahydrofuran and also the mixtures thereof with aqueous buffers or water. These solvents, which present a risk to health, have to be removed prior to sensory evaluation. The use of time-intensive, extractive or thermal processes to remove the solvents presenting a risk to health frequently leads to a change in the composition of the fractions obtained by liquid chromatography. This is caused by thermal and/or oxidative influences during the treatment. The sensory properties of fractions treated in this way often differ considerably from the original compositions. WO 2006/111476 has already disclosed a process for the separation of olfactorily and/or gustatorially active components by means of high temperature liquid chromatography (HTLC) and also a process for the direct tasting of the fractions obtained by this process. A drawback of this process is the fact that the eluate of a separated aroma substance can comprise, owing to its distribution properties, an ethanol content of much more than 15% by weight based on the total weight of the eluate. On the one hand an ethanol content of more than 15% by weight based on the total weight of the eluate can however lead to influencing of the odour or taste receptors of a test subject, and thus to misinformation concerning the sensory properties, and on the other hand an ethanol content of more than 15% by weight can lead, on tasting of conventionally 5 ml per sample, to temporary impairment of the test subject's state of mind when tasting a plurality of samples, so that merely a few tastings can be carried out in a day.

DE 3 941 533 A1 describes an interface for coupling a liquid chromatography system to a solid or gas phase detector. The interface according to the invention comprises a nebuliser with thermospray or other spray technology, coupled to an evaporation chamber and an industrial detector, for example a mass detector, wherein even in the nebuliser the temperature is increased to more than 100° C., to produce particularly small drops of aerosol and thus to attain a large surface area, so that in the subsequent evaporation step almost the entire solvent is evaporated and a “dry” aerosol comprising the inert gas and the particles of interest is forwarded to the gas phase or solid phase detector for analysis. A drawback of this process is the fact that no sensory assessment is carried out with regard to the odour or taste properties of the individual aroma substances.

Processes for evaluation by electronic sensor systems (“in vitro”) are also known. A drawback of these is the frequently missing transferability to the taste and/or odour impressions of human beings.

The present invention is therefore based on the object of providing a device and a process for producing a flavour substance preparation comprising or consisting of one or more flavouring substances, water and ethanol for the (i) real-time (“on-line”) or synchronous sensory assessment by a test subject (“in vivo”) with regard to taste (tasting), wherein the flavouring substance preparation produced (ii) has a low risk of misjudgement of the taste impression owing to impairment of the taste receptors of the tongue by the ethanol content and/or (iii) allows tasting of two, three, four, five and more samples in a day, without causing temporary impairment of the test subject's state of mind owing to the alcohol content. Furthermore, it is the object of the present invention to produce a flavouring substance preparation of this type rapidly and simply, wherein the flavouring substances contained are (substantially) not impaired with regard to their taste impression.

### SUMMARY OF THE INVENTION

The objects of the present invention are achieved completely or partly by the subject matters according to the invention according to the independent claims.

Accordingly, the object of the present invention is achieved by a device (1) for reducing an ethanol content in a liquid preparation with a nebulising means (2), a mixing chamber (3), a separation chamber (4) and a condensation chamber (5), characterised in that
the nebulising means (2) comprises a liquid inlet (21), a gas inlet (22) and a connection to the mixing chamber (3) or consists thereof and the nebulising means (2) is operated at a temperature of ≦100° C., so that there can be supplied to the nebulising means (2) a liquid comprising water and ethanol having an ethanol content of >15% by weight based on the total weight of the liquid by means of the liquid inlet (21) and an inert gas by means of the gas inlet (22) and the nebulising means (2) is capable of nebulising the liquid and the inert gas to form an aerosol and thus of supplying them into the mixing chamber (3),
the mixing chamber (3) comprises an evaporation means (31) and a connection to the separation chamber (4) or consists thereof, wherein the mixing chamber (3) is arranged after the nebulising means (2) and connected thereto in such a way that the aerosol formed by means of the nebulising means (2) can be supplied into the mixing chamber (3) and the evaporation means (31) is capable of converting a part or the ethanol of the supplied aerosol partly or completely into the gas phase,
the separation chamber (4) comprises a gas outlet (41) and a connection to the condensation chamber (5) or consists thereof, wherein the separation chamber (4) is arranged after the mixing chamber (3) and connected thereto in such a way that the ethanol converted into the gas phase and the remaining aerosol can be supplied into the separation chamber (4) and a part or the ethanol gas phase can be removed from the device through the gas outlet (41) and
the condensation chamber (5) comprises a liquid outlet (51) and a condensation means (52) or consists thereof, wherein the condensation chamber (5) is arranged after the separation chamber (4) and connected thereto in such a way that the aerosol remaining in the separation chamber (4) after partial or complete removal of the ethanol gas phase can be supplied into the condensation chamber (5) and the condensation means (5) is capable of wholly or partly condensing the supplied aerosol and the condensate having an ethanol content of ≦15% by weight based on the total weight of the condensate can be removed from the condensation chamber (5) through the liquid outlet (51).

A second subject matter according to the invention relates to a process for reducing an ethanol content in a flavouring substance preparation comprising or consisting of one or more flavouring substances, water and ethanol comprising or consisting of the following steps:
a) providing a liquid flavouring substance preparation which comprises one or more flavouring substances, water and ethanol having an ethanol content of >15% by weight based on the total weight of the flavouring substance preparation or consists thereof, and also an inert gas,
b) supplying the liquid flavouring substance preparation through a liquid inlet (21) and the inert gas through a gas entrance (22) into a nebulising means (2) by means of which the flavouring substance preparation and the inert gas are nebulised to form an aerosol,
c) supplying the aerosol formed in step b) into a mixing chamber (3) which is connected to the nebulising means (2) and has evaporation means (31) and converting a part of or the entire ethanol component of the aerosol by means of the evaporation means (31) into a gas phase,
d) supplying the aerosol formed in step c) into a separation chamber (4) which is connected to the mixing chamber (3) and has a gas outlet (41) and removing a part of or the entire gaseous ethanol component from the device through the gas outlet (41),
e) supplying the aerosol formed in step d) into a condensation chamber (5) which is connected to the separation chamber (4) and has a liquid outlet (51) and condensation means (52) and condensing the aerosol by means of the condensation means (52) to form a flavouring substance concentrate comprising or consisting of the flavouring substance or substances, water and ethanol having an ethanol content of ≦15% by weight based on the total weight of the flavouring substance concentrate and
f) removing the flavouring substance concentrate formed in step e) from the condensation chamber (5) through the liquid outlet (51).

A third subject matter according to the invention relates to a process for sensory assessment of one or more flavouring substances comprising or consisting of the following steps:
i. producing a liquid flavouring substance concentrate comprising or consisting of one or more flavouring substances, water and ethanol having an ethanol content of ≦15% by weight based on the total weight of the flavouring substance concentrate in accordance with one of the processes of the second subject matter according to the invention and also the preferred configurations and
ii. supplying the flavouring substance concentrate into the mouth of a test subject for sensory assessment.

A fourth subject matter according to the invention relates to a use of a device according to the invention for producing a liquid flavouring substance concentrate comprising or consisting of one or more flavouring substances, water and ethanol having an ethanol content of ≦15% by weight based on the total weight of the flavouring substance concentrate.

The percentages by weight (% by weight) in relation to the subject matters according to the invention are each based on the total weight of the respective liquid preparation.

The subject matters according to the invention also comprise in any conceivable combination the preferred configurations, such as they are represented in the subsequent description, exemplary embodiments, figures and dependent claims.

### DESCRIPTION OF THE FIGURES

The figures show both a schematic construction of a device (1) according to the invention and the device (1) according to the invention in connection with the preceding and subsequent systems.

FIG. 1 is a schematic construction of a device (1) according to the invention;

FIG. 2 is a schematic construction of a device (1) according to the invention coupled to a liquid chromatography system (6); and

FIG. 3 is a schematic construction of a device (1) according to the invention coupled to a sample vessel (61) containing a flavouring substance sample.

### LIST OF REFERENCE NUMERALS

(1) Device according to the invention
(2) Nebulising means
(21) Liquid inlet nebulising means
(22) Gas inlet nebulising means
(3) of a mixing chamber
(31) Evaporation means
(32) Inner wall of the mixing chamber
(33) Outer wall of the mixing chamber
(34) Inlet for a temperature control medium
(35) Outlet for a temperature control medium
(36) Gas inlet mixing chamber
(4) Separation chamber
(41) Gas outlet
(411) End of the gas outlet in the lower region inside the separation chamber
(412) End of the gas outlet in the region outside the separation chamber/device
(42) Swirling means
(43) Wall of the separation chamber
(5) Condensation chamber
(51) Liquid outlet
(52) Condensation means
(53) Inner wall of the mixing chamber
(54) Outer wall of the mixing chamber
(55) Inlet for a temperature control medium
(56) Outlet for a temperature control medium
(6) Liquid chromatography system
(61) Sample vessel comprising flavouring substance sample
(62) Pump
(7) Inert gas container
(8) Detector means

### DETAILED DESCRIPTION OF THE INVENTION

The present invention is based on the recognition that, using the device according to the invention, a liquid flavouring substance concentrate comprising or consisting of one or more flavouring substances, water and ethanol having an ethanol content of ≦15% by weight based on the total weight of the flavouring substance concentrate can be produced simply and rapidly and thus allows (i) a real-time (“on-line”) or synchronous sensory assessment by a test subject (“in vivo”). The flavouring substance concentrate which can be produced in accordance with the invention has in addition, (ii) in the in vivo sensory assessment, a low risk of a misjudgement of the sensory properties of the flavouring substance or substances as, owing to the ethanol content of ≦15% by weight based on the total weight of the flavouring substance concentrate, the taste receptors are not (significantly) impaired and (iii) allows, in an in vivo sensory assessment, a tasting of two, three, four or more samples (conventionally ≦5 ml/sample) of one or more flavouring substance concentrates in a day, without temporarily impairing the test subject's state of mind.

In the sense of the present invention, the term “flavouring substance” encompasses a sensorially active substance which brings about, i.e. itself imparts, a taste impression or changes, i.e. modifies or intensifies, the taste perception of another substance owing to its own taste, wherein the substance has a vapour pressure of <0.01 Pa at 25° C.

In the sense of the present invention, the term “flavouring substance preparation” means a liquid preparation comprising or consisting of one or more flavouring substances, water and ethanol having an ethanol content of >15% by weight based on the total weight of the flavouring substance preparation. Preference is given to flavouring substance preparations of the type in which the flavouring substance or substances has/have a proportion of ≦5% by weight, preferably ≦2.5% by weight, more preferably ≦1% by weight and at least 10⁻¹²% by weight and the water/ethanol mixture has a proportion of equal to or greater than 95% by weight, preferably equal to or greater than 97.5% by weight, more preferably equal to or greater than 99% by weight, based in each case on the total weight of the flavouring substance preparation. The ethanol content in the water/ethanol mixture lies preferably in the range of from 16 to 95% by weight, more preferably in the range of from 25 to 90% by weight, particularly preferably in the range of from 40 to 85% by weight, most particularly preferably in the range of from 50 to 80% by weight, based in each case on the total weight of the water/ethanol mixture. The higher the proportion of ethanol in the water/ethanol mixture of the flavouring substance preparation is, the less finely does the flavouring substance preparation need, at a comparable temperature, to be nebulised to obtain a reduction according to the invention of the ethanol content.

The water/ethanol mixture to be used in accordance with the invention of the flavouring substance preparation according to the invention can additionally comprise or consist of one or more constituents selected from the group consisting of propylene glycol, glycerol, triacetin (glycerol triacetate), physiologically compatible salts (for example sodium chloride), physiologically compatible acids (for example phosphoric acid, acetic acid), physiologically compatible buffer substances (for example sodium phosphates, sodium acetate), oils and fats, wherein the concentration of the constituent or constituents in the water/ethanol mixture is expediently in each case selected in such a way that the flavouring substance preparation according to the invention as a whole continues not to pose a threat to health.

In so far as the above-preferred water/ethanol mixture of the flavouring substance preparation according to the invention comprises physiologically compatible salts (for example sodium chloride), physiologically compatible acids (for example phosphoric acid, acetic acid) and/or physiologically compatible buffer substances (for example sodium phosphates, sodium acetate), the overall proportion of these salts, acids and/or buffer substances lies preferably at ≦10% by weight, preferably at ≦5% by weight, particularly preferably in the range of from 0.001 to 2% by weight, based in each case on the total weight of the flavouring substance preparation.

In the sense of the present invention, the “flavouring substance concentrate” is produced from the flavouring substance preparation according to the invention and comprises or consists of one or more flavouring substances, water and ethanol having an ethanol content of ≦15% by weight, preferably in the range of from 0 to 10% by weight, more preferably in the range of from 0.1 to 8% by weight, particularly preferably in the range of from 1 to 5% by weight, based in each case on the total weight of the flavouring substance concentrate. Accordingly, the flavouring substance concentrate according to the invention is present in reduced form compared to the flavouring substance preparation according to the invention in relation to the ethanol content. Furthermore, the additional constituents of the flavouring substance preparation can also be present in the flavouring substance concentrate according to the invention.

In the sense of the present invention, the term “liquid preparation”, which can be supplied into the device (1) according to the invention, means a liquid preparation comprising water and ethanol having an ethanol content of >15% by weight based on the total weight of the liquid preparation. The ethanol content in the liquid preparation lies preferably in the range of from 16 to 95% by weight, more preferably in the range of from 25 to 90% by weight, particularly preferably in the range of from 40 to 85% by weight, most particularly preferably in the range of from 50 to 80% by weight, based in each case on the total weight of the liquid preparation. In a preferred configuration, the liquid preparation is the liquid flavouring substance preparation according to the invention. The “condensate” to be obtained from the “liquid preparation” by means of the device according to the invention comprises or consists accordingly of water and ethanol having an ethanol content of ≦15% by weight, preferably in the range of from 0 to 10% by weight, more preferably in the range of from 0.1 to 8% by weight, particularly preferably in the range of from 1 to 5% by weight, based in each case on the total weight of the condensate. In a preferred configuration, the condensate according to the invention is the flavouring substance concentrate according to the invention.

In the sense of the present invention, the term “inert gas” means that the supplied inert gas does not chemically change the “liquid preparation” according to the invention, in particular the “flavouring substance preparation” according to the invention. Inert gases are preferably selected from the group consisting of: nitrogen, helium or argon. Particularly preferably, nitrogen is used in accordance with the invention as the inert gas.

In the sense of the present invention, the term “sensory assessment” means that a sample, conventionally ≦5 ml, of a flavouring substance concentrate produced in accordance with the invention is taken by a test subject into his mouth and if appropriate swallowed, wherein the sample is assessed gustatorially and/or retronasally with regard to the sensory impressions, for example umami, salty, sour, sweet, bitter, or prickling, cooling, sharp, burning or electrifying (“tingling”) effects.

In the sense of the present invention, a “liquid chromatography system (6)” to be used in accordance with the invention is a device which is capable of carrying out a liquid chromatography [liquid chromatography (LC chromatography), preferably high pressure liquid chromatography (HPLC chromatography) and in particular high temperature liquid chromatography (HTLC chromatography)] of a flavouring substance preparation with detection of the flavouring substance or substances.

Suitable separating techniques for the liquid chromatography systems (6) to be used in accordance with the invention include: reversed phase chromatography (RP), normal phase chromatography (NP), size exclusion chromatography (SEC) and hydrophilic interaction chromatography (HILIC).

Conventionally, the detectors are selected from the group consisting of mass selective detectors (MS), ultraviolet detectors (UV), diode array detectors (DAD), refractive index detectors (RI), evaporative light scattering detectors (ELSD), fluorescence detectors (FLD) or charged aerosol detectors (CAD).

Preferably, an HPLC liquid chromatography system (6) comprises or consists of a degasser (for example 3315, from ERC), two pumps (for example Sun Flow 100, from Sunchrom), a mixer (for example a dynamic/static mixer, from Sunchrom), an autosampler (for example Midas, from Spark), a chromatography column (for example a Hamilton PRP-1 250×10 mm column) and a detector (preferably a diode array detector, for example Spectra Flow 600, from Sunchrom). In the case of an HTLC liquid chromatography system to be used in accordance with the invention, the chromatography column is conventionally arranged in a column oven suitable for setting the required temperature in the column (for example Polaratherm, from Selerity).

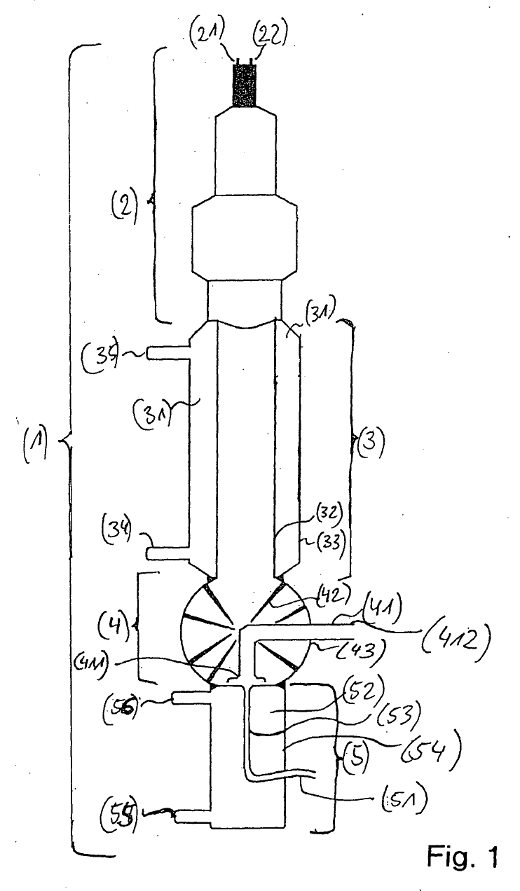

FIG. 1 shows a schematic construction (on a scale of 1:2, i.e. the device is twice as large as the illustration in FIG. 1) of a device (1) according to the invention which is capable of reducing in accordance with the invention an ethanol content of a liquid preparation according to the invention, preferably a flavouring substance preparation according to the invention.

The liquid preparation, preferably a flavouring substance preparation, is supplied through the liquid inlet (21) into the nebulising means (2). Additionally, an inert gas is supplied through the gas inlet (22) (substantially) at the same time for the introduction of liquid into the nebulising means (2). By means of the nebulising means (2), the supplied liquid and the supplied inert gas are nebulised to form an aerosol and supplied into the mixing chamber (3) as aerosol.

According to the invention, the nebulising means (2) is operated at a temperature of ≦100° C., preferably ≦75° C., more preferably in a range of from 10 to 40° C., particularly preferably in a range of from 15 to 30° C. and most particularly preferably in a range of from 20 to 25° C. As a result of the fact that the working temperature in the nebulising means (2) is operated in accordance with the invention in the region of ≦100° C., for example the risk of delays in boiling of the water/ethanol mixture is reduced. In addition, the drop size of the aerosol can be controlled by adjusting the temperature. In the case of drops of the aerosol having an on average relatively small diameter, the surface area of the liquid in the aerosol is larger than in the case of drops having an on average relatively large diameter. The larger the surface area of the drops in the aerosol, the higher the proportion of the evaporation of the ethanol or the ethanol/water mixture in the aerosol in the subsequent evaporation step will be.

The supplied liquid and the supplied inert gas are supplied into the nebulising means at a conventional flow speed or a conventional pressure, so that on the one hand the nebulising to form an aerosol is facilitated and on the other hand the aerosol formed can be conveyed through the device (1) according to the invention. Flow speeds and pressures can depend on the configuration of the nebulising means (2) and vary accordingly.

Nebulising means (2) to be used in accordance with the invention are preferably nebulising means for, for example, evaporative light scattering detectors (ELSD) which are illustrated for example in U.S. Pat. No. 7,290,723, U.S. Pat. No. 6,528,018, U.S. Pat. No. 6,485,689, U.S. Pat. No. 6,362,880, U.S. Pat. No. 6,229,605, U.S. Pat. No. 6,151,113 and U.S. Pat. No. 6,122,055, wherein the configurations concerning the respective nebulising means from the documents mentioned hereinbefore are incorporated into the present description, and also thermospray nebulisers which are illustrated for example in DE 3 941 533 A1, U.S. Pat. No. 4,730,111 and U.S. Pat. No. 4,629,478, wherein the configurations concerning thermospray nebulisers from the respective documents mentioned hereinbefore are incorporated into the present description. Particular preference is given to nebulising means for evaporative light scattering detectors, for example the nebulising means from Sedere for the flow range of from 1-5 ml/min.

The preferred nebulising means from Sedere comprises conventionally a gas inlet (22) with a controller for adjusting a working pressure in the nebulising means (2) in the range of from 0.5 to 5 bar, preferably in the range of from 1 to 4 bar, more preferably 2 to 3 bar and most particularly preferably at 3 bar.

Additionally or alternatively, the liquid inlet (21) comprises conventionally a controller for adjusting the flow speed of the liquid into the nebulising means (2) in the range of from 1 to 5 ml/min, preferably 2 to 4 ml/min, particularly preferably 3 ml/min.

The preferred flow speeds and working pressures illustrated hereinbefore allow particularly preferred nebulising to form an aerosol and preferred subsequent forwarding of the aerosol formed through the device (1) according to the invention.

In a further preferred configuration, the device (1) according to the invention is, additionally or alternatively to the foregoing preferred configurations, constructed perpendicularly to the centre of the earth, so that the nebulising means (2) is arranged at the upper end, i.e. at the end furthest away from the centre of the earth, of the device (1) according to the invention, and the condensation chamber (5) is arranged at the lower end, i.e. at the end closest to the centre of the earth, of the device (1) according to the invention. This construction is advantageous, as gravity additionally contributes to the present working pressure for forwarding the aerosol through the device (1) according to the invention and thus the flow speed and/or the working pressure can be reduced.

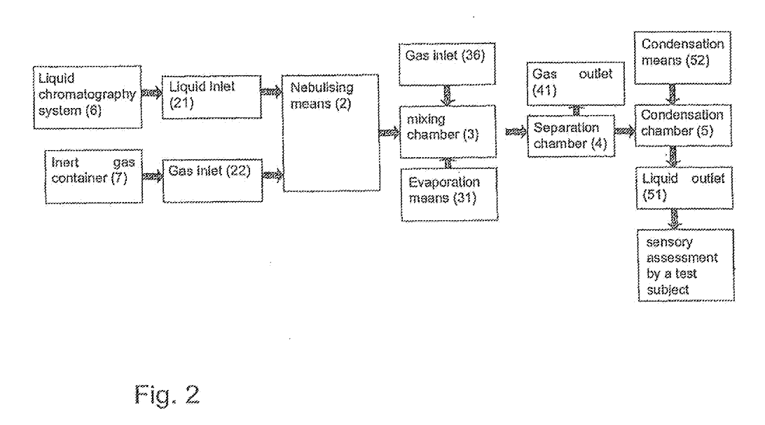

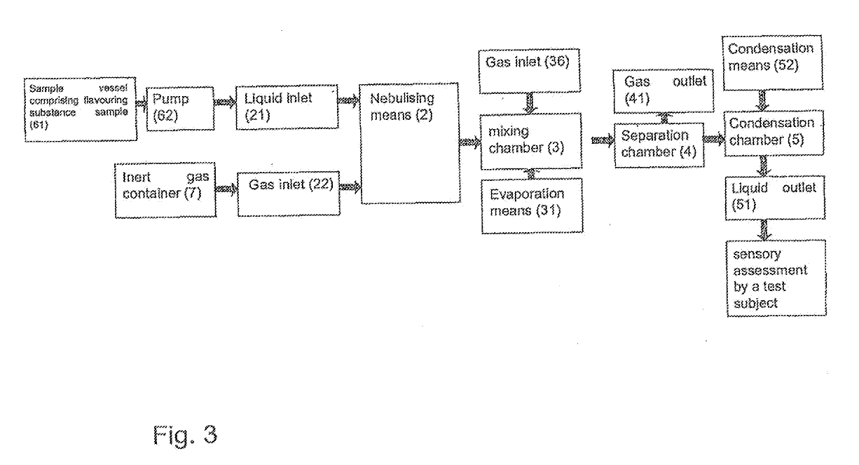

In a further preferred configuration, the device (1) according to the invention is, additionally or alternatively to the foregoing preferred configurations, characterised in that the mixing chamber (3) additionally comprises a gas inlet (36) (see FIGS. 2 and 3) for directly supplying an inert gas into the mixing chamber (3). This preferred configuration allows the flow speed of the aerosol to be controlled additionally to the controllers of the liquid inlet (21) and the gas inlet (22).

In a further preferred configuration, the device (1) according to the invention is, additionally or alternatively to the foregoing preferred configurations, characterised in that the evaporation means (31) is configured as a temperature controller in the range of from 30 to 100° C., preferably 50 to 95° C., more preferably 70 to 90° C., particularly preferably 75 to 85° C. and most particularly of 80° C. The temperature is conventionally adjusted in such a way that a sufficient quantity of ethanol is converted into the gas phase, so that the ethanol component removed in the separation chamber (4) from the device (1) is sufficient, so that the condensate removed from the condensation chamber (5) by means of the liquid outlet (51) has an ethanol content of ≦15% by weight, preferably in the range of from 0 to 10% by weight, more preferably in the range of from 0.1 to 8% by weight, particularly preferably in the range of from 1 to 5% by weight, based in each case on the total weight of the condensate. Preferably, the proportion of ethanol which passes in the mixing chamber (3) into the gas phase lies therefore at ≧50% by weight, more preferably in the range of from 60 to 100% by weight, particularly preferably in the range of from 70 to 95% by weight, based in each case on the total weight of that of the supplied flavouring substance preparation.

In a further preferred configuration, the device (1) according to the invention is, additionally or alternatively to the foregoing preferred configurations, characterised in that the mixing chamber (3) is configured as a tube, i.e. with a (substantially) circular cross section of conventionally 50 mm, preferably 35 mm and particularly preferably 25 mm, and with a length of conventionally 150 mm, preferably 120 mm and particularly preferably 90 mm. Particularly preferably, this tube has a uniform cross section and/or is not curved. This preferred configuration is advantageous as, owing to the tube shape, the dead volume is minimised and thus leads to a low turbulence flow.

In a further preferred configuration, the device (1) according to the invention is, additionally or alternatively to the foregoing preferred configurations, characterised in that the mixing chamber (3) is configured as a double-walled tube having around its first inner wall (32) a second outer wall (33) which is set apart therefrom and has an inlet (34) and an outlet (35) for a temperature control medium. In this preferred configuration, the evaporation means (31) consists in the double-walled tube configuration (heating jacket), in which the temperature control medium, conventionally a liquid, preferably a liquid selected from the group consisting of water or mineral oil as the heat transfer medium and water, ethylene glycol or mixtures of water and ethylene glycol as the cold transfer medium, is heated to the desired temperature and supplied through the inlet (34) into the space between the first inner wall (32) and the second outer wall (33), which is set apart therefrom, and is if appropriate removed from the outlet (35). This preferred configuration allows the mixing chamber (3) to be able to be tempered (substantially) constantly, as new tempered temperature control medium can always be supplied to the heating jacket or temperature control medium which has already been used can be heated, after issuing from the outlet (35), back to the starting temperature.

In an alternative configuration, an evaporation means to be used in accordance with the invention can be a thermoelectric plate (Peltier element), an electric strip heater, an electric heating jacket or an electric heating tube.

In a further preferred configuration, the device (1) according to the invention is, additionally or alternatively to the foregoing preferred configurations, characterised in that the separation chamber (4) has an at least equally large or larger cross section, i.e. a (substantially) circular cross section, compared to the mixing chamber (3), wherein the cross section of the separation chamber (4) lies conventionally in the region of 100 mm, preferably 70 mm, particularly preferably 50 mm and the length of the separation chamber (4) conventionally in the region of 150 mm, preferably 100 mm and particularly preferably 65 mm. In a most particularly preferred configuration according to the invention, the separation chamber (4) is configured spherically. A change in the cross section leads conventionally to diffusion of the evaporated aerosol, so that flow turbulences are produced and swirling in the separation chamber (4) is supported. This likewise supports the separation of the gas component and the liquid aerosol component, so that the gaseous ethanol component can be removed in an improved manner through the gas outlet (41) from the separation chamber and thus from the device (1) according to the invention. In a further preferred configuration, the swirling of the aerosol in the separation chamber (4) is further supported by one or more swirling means (42), preferably rod or mandrel-like means which are connected to the separation chamber wall (43) and extend into the interior of the separation chamber (4).

In a further preferred configuration, the device (1) according to the invention is, additionally or alternatively to the foregoing preferred configurations, characterised in that the gas outlet (41) is configured as a tube, i.e. with a (substantially) circular cross section, which runs through an outer wall (43) of the separation chamber (4) into a space outside the device (1), wherein there are arranged one end of the tube (411) inside the separation chamber (4) in a region opposing the connection to the mixing chamber (3) and a second end of the tube (412) in the space outside the device (1). In a further preferred configuration, the device (1) according to the invention is, additionally or alternatively to the foregoing preferred configurations, characterised in that the gas outlet (41) is arranged in the constructed state at the lower end of the separation chamber (5) and runs in a region above the lower end through the outer wall (43) of the separation chamber (4) into the space outside the device (1).

It is preferred for the gas outlet (41) to be arranged at the lower end of the separation chamber (4), i.e. in locational proximity to the condensation chamber (5) since, as a result of the drop in temperature, the liquid drops of the aerosol predominantly condense and can be discharged through the liquid outlet (51) as condensate, while the predominantly separated gas component of the aerosol can be removed through the gas outlet (41) from the device (1).

The gas outlet (41) is preferably a glass tube, having a cross section of from 5 to 15 mm, more preferably about 10 mm, and is conventionally arranged less than 10 mm, preferably less than 5 mm, most particularly preferably about 3 mm, above the transition to the condensation chamber (5). In a further preferred configuration, the end of the tube (411) is configured in a funnel-shaped manner. The funnel-shaped configuration of the opening (411) prevents turbulences in the condensation zone, so that the condensate can drop uniformly out of the liquid outlet (51).

In a preferred configuration, the gas outlet is arranged not linearly but rather in an angled manner, preferably at a 90° angle. In this case, the lengths of the gas outlet tube (41) perpendicularly (perpendicular tube) and horizontally (transverse tube) relative to the centre of the earth are conventionally of roughly equal length; preferably, the perpendicular tube is approximately 27 mm long and is at a distance of 3 mm from the condensation chamber (5) and the transverse tube is preferably 30 mm long.

In a further preferred configuration, the device (1) according to the invention is, additionally or alternatively to the foregoing preferred configurations, characterised in that the separation chamber (4) is operated at ambient temperature and is not cooled in accordance with the condensation chamber (5). This configuration is preferred because the coupling of the heated mixing chamber (3) and the cooled condensation chamber (5) via the separation chamber (4) to be used in accordance with the invention interposes a “temperature buffer” which reduces material damage to the device (1) owing to the drop in temperature.

In a further preferred configuration, the device (1) according to the invention is, additionally or alternatively to the foregoing preferred configurations, characterised in that the condensation chamber (5) is a tube, i.e. has a (substantially) circular cross section. As stated hereinbefore with regard to the mixing chamber (3), a tube has a low dead volume.

In a further preferred configuration, the device (1) according to the invention is, additionally or alternatively to the foregoing preferred configurations, characterised in that the tube of the condensation chamber (5) has a smaller cross section compared to the separation chamber (4) and/or the mixing chamber (3), i.e. conventionally a cross section in the region of 8 mm, preferably 5 mm and particularly preferably 2 mm and a length of conventionally 80 mm, preferably 50 mm and particularly preferably 35 mm. This reduction in cross section leads to a contraction of the aerosol which is obtained in the separation chamber (4) and forwarded into the condensation chamber (5), so that the effect of the condensation means (52) is supported.

In a further preferred configuration, the device (1) according to the invention is, additionally or alternatively to the foregoing preferred configurations, characterised in that the condensation means (52) is configured as a temperature controller in the range of from 0 to 60° C., preferably 5 to 50° C., more preferably 10 to 40° C., particularly preferably 15 to 25° C. and most preferably of 20° C. Owing to these temperature ranges, the condensation of the aerosol relative to the flavouring substance concentrate according to the invention is supported.

In a further preferred configuration, the device (1) according to the invention is, additionally or alternatively to the foregoing preferred configurations, characterised in that the condensation chamber (5) is configured as a double-walled tube having around its first inner wall (53) a second outer wall (54) which is set apart therefrom and has an inlet (55) and an outlet (56) for a temperature control medium, conventionally a liquid, preferably a liquid selected from the group consisting of water or mineral oil as the heat transfer medium and water, ethylene glycol or mixtures of water and ethylene glycol as the cold transfer medium. As stated hereinbefore with regard to the mixing chamber (3) according to the invention, this preferred configuration allows the condensation chamber (5) to be tempered (substantially) constantly.

In an alternative configuration, a condensation means (52) to be used in accordance with the invention can be a thermoelectric plate (Peltier element), an electric tempering strip, an electric tempering jacket or an electric tempering tube.

In a further preferred configuration, the device (1) according to the invention is, additionally or alternatively to the foregoing preferred configurations, characterised in that the liquid outlet (51) is arranged at the lower end of the condensation chamber (5). This preferred configuration allows the condensate formed to be able to be removed by means of gravity through the liquid outlet (51) from the condensation chamber (5) and thus from the device (1) according to the invention. Nevertheless, additionally, further means, in particular pumps (for example suitable hose pumps or LC pumps—not shown), could also be connected downstream of the condensation chamber (5) to remove the condensate from the device (1).

The device (1) according to the invention can be made of any suitable material, preferably of one or more materials from the group consisting of glass, metal, noble metal, steel or plastics material (for example polytetrafluoroethylene or polyether ether ketone), particularly preferably glass, wherein the individual chambers and means are connected to one another in such a way that from the device according to the invention (1), apart from at the outlet points provided, in particular the gas outlet (41) and the liquid outlet (51), (substantially) neither the supplied inert gas and/or the supplied liquid preparation, preferably the liquid flavouring substance preparation, escapes from the device (1) according to the invention. In a particularly preferred configuration, the device (1) according to the invention is constructed, additionally or alternatively to the foregoing particularly preferred configurations, partly or completely from heat resistant glass.

In a further preferred configuration, the condensate removed from the device (1) according to the invention can, additionally or alternatively to the foregoing preferred configurations, be separated into two, three or more eluate fractions, wherein the fractions are particularly preferably separated in such a way that one fraction comprises substantially one flavouring substance. To determine a flavouring substance concentrate fraction containing only one flavouring substance, use may be made of one or more, identical or different, suitable detectors (not shown), preferably selected from the group consisting of mass selective detectors (MS), ultraviolet detectors (UV), diode array detectors (DAD), refractive index detectors (RI), evaporative light scattering detectors (ELSD), fluorescence detectors (FLD) or charged aerosol detectors (CAD), which are arranged before and/or after the device (1) according to the invention. This preferred configuration allows sensory assessment of the isolated flavouring substance and thus estimation of the sensory contribution of the flavouring substance in an aroma preparation.

FIG. 2 shows a schematic construction of a device (1) according to the invention, which likewise comprises the above-illustrated preferred configurations in all conceivable combinations, coupled to a liquid chromatography system (6) to be used in accordance with the invention.

The preferred configuration of the device (1) according to the invention can be connected in a preferred configuration to the liquid chromatography systems (6), which can be used in accordance with the invention, of different separation techniques directly, i.e. via the liquid inlet (21), so that the eluate can be supplied through the liquid inlet (21) into the first nebulising means (2).

The coupling of the device (1) according to the invention is preferred, on the one hand, because through the separation by liquid chromatography of a liquid flavouring substance sample comprising two, three or more flavouring substances, a separation in time of the flavouring substances through the eluant, water and ethanol used is already carried out, if appropriate in gradient mode, wherein the eluate from a separation by liquid chromatography corresponds to the flavouring substance preparation according to the invention. On the other hand, the liquid chromatography system (6) to be used in accordance with the invention already comprises a detector means which is accordingly connected upstream of the device (1) according to the invention, thus allowing a better estimation as to when the flavouring substance or substances is/are to be expected, after passing through the device (1) according to the invention, in the flavouring substance concentrate produced.

As a result of the compact design of the device (1) according to the invention and preferably on additional supplying of inert gas directly into the mixing chamber (3) through a gas inlet (36), the flow speed of an LC eluate as the liquid flavouring substance preparation or the flow speed of the aerosol, i.e. the duration of the conveyance through the device (1) according to the invention, is adjusted in such a way that effective separation by liquid chromatography remains ensured and thus a real-time (“online”) or synchronous sensory assessment with regard to the taste of a single flavouring substance and thus to the sensory contribution of the flavouring substance by a test subject is facilitated. Conventionally, at a pressure of 3 bar on the nebulising means (2), the aerosol/gas speed is ˜65 ml/s. The pass duration of the aerosol varies as a function of the temperatures (evaporation/condensation) in the device (1), between fractions of seconds to 1, 2 or a few seconds.

FIG. 3 shows a schematic construction of a device (1) according to the invention, which likewise comprises the above-illustrated preferred configurations in all conceivable configurations, coupled to a sample vessel (61) containing a flavouring substance preparation according to the invention comprising preferably a flavouring substance, which are connected to one another in such a way that this flavouring substance preparation is supplied, if appropriate by means of a suitable conventional pump (62), for example an LC pump or hose pump, through the liquid inlet (21) into the first nebulising means (2). A detector means (8) can preferably be interposed between the sample vessel (61) and the liquid inlet (21) in order to be able better to judge when the flavouring substance or substances is/are forwarded through the device (1) according to the invention.

Conventionally, the detectors are selected from the group consisting of mass selective detectors (MS), ultraviolet detectors (UV), diode array detectors (DAD), refractive index detectors (RI), evaporative light scattering detectors (ELSD), fluorescence detectors (FLD) or charged aerosol detectors (CAD).

### EXEMPLARY EMBODIMENT

The present invention will be described hereinafter by way of an exemplary embodiment which does not, however, limit the scope of protection of the subject matters according to the invention.

### Example 1

### Identification of a Sweet-Tasting Compound Consisting of Lippia dulcis

An extract of Lippia dulcis (Aztec sweet herb) was separated by means of high temperature liquid chromatography (HTLC) using a water/ethanol gradient. The eluate was concentrated using the process according to the invention and sensorially assessed.

### 1. Production of a Sample Solution

A sample solution of Lippia dulcis was produced by dissolving/suspending 500 mg of Lippia dulcis in a mixture of water and ethanol (1:1/v:v), with subsequent membrane filtration of the non-dissolved residue.

### 2. Carrying Out Liquid Chromatography of the Sample Solution

The HTLC device used, consisting of a degasser (3315, from ERC), two pumps (Sun Flow 100, from Sunchrom), a mixer (dynamic/static mixer, from Sunchrom), an autosampler (Midas, from Spark), a column oven (Polaratherm, from Selerity) and a diode array detector (Spectra Flow 600, from Sunchrom), was equipped with a Hamilton PRP-1 250×10 mm column and conditioned with a flow of 2 ml/min of an eluant, consisting of 50% by weight of ethanol and 50% by weight of water.

To apply the samples into the conditioned HTLC device used, 100 μl of the clear solution produced were injected using the autosampler.

Once conditioning has been carried out, the chromatographic analysis takes place in accordance with the following programme:

| | | | | Elution | Temperature of | Temperature | Diode array |
|---|---|---|---|---|---|---|---|
| Time | Flow | Water | Ethanol | gradient | the column oven | gradient | detector |
| [min] | [ml/min] | [%] | [%] | [%/min] | [° C.] | [° C./min] | (DAD) |
| 0 | 2 | 50 | 50 | Isocratic | 40 | Isothermal | 210 nm |
| 6 | 2 | 50 | 50 | Isocratic | 40 | 20 | 210 nm |
| 10 | 2 | 50 | 50 | 1.25 | 120 | Isothermal | 210 nm |
| 50 | 2 | 0 | 100 | Isocratic | 120 | Isothermal | 210 nm |
| 60 | 2 | 0 | 100 | Isocratic | 120 | Isothermal | 210 nm |

The changes in temperature, flow and eluant composition during the programme take place linearly.

The eluate is introduced subsequently to the chromatographic separation under the following conditions via the liquid inlet (21) directly into the nebulising means (2) of the device (1) according to the invention:

| Gas pressure | Flow in | Temperature of the | Temperature of the |
|---|---|---|---|
| in the gas inlet | the liquid | evaporation means | condensation means |
| (22) | inlet (21) | (31) | (51) |
| 3 bar | 2 ml/min | 80° C. | 30° C. |

The sensorially active compound phyllodulcin, which is contained in Lippia dulcis, eluted after separation by liquid chromatography at an ethanol concentration of approx. 75%. The eluate was supplied at a pressure of 3 bar via the gas inlet (22) into the nebulising means (2) according to the invention and supplied as a nebulised aerosol into the mixing chamber (3), which had been preheated to 80° C., of the device (1) according to the invention. The condensate or flavouring substance concentrate according to the invention obtained at the liquid outlet (51) of the condensation chamber (5) had an ethanol content of ≦15% of ethanol.

The flavouring substance concentrates according to the invention of the compounds separated by liquid chromatography were directly sensorially assessed by a panel made up of trained test subjects. Phyllodulcin was sensorially detected by a typical strong sweet taste. A direct sensory assessment of the compounds separated by liquid chromatography, without using the device according to the invention and the process according to the invention, would have entailed in the described example a high risk of misjudgement of the taste impression owing to the impairment of the taste receptors of the tongue by the ethanol content and/or a tasting of a plurality of samples in succession would have led to temporary impairment of the test subject's state of mind, likewise owing to the alcohol content.

### SPECIFIC EMBODIMENTS

Specific embodiment one comprises a device (1) for reducing an ethanol content in a liquid preparation with a nebulising means (2), a mixing chamber (3), a separation chamber (4) and a condensation chamber (5), characterised in that
   the nebulising means (2) comprises a liquid inlet (21), a gas inlet (22) and a connection to the mixing chamber (3) or consists thereof and the nebulising means (2) is operated at a temperature of ≦100° C., so that there can be supplied to the nebulising means (2) a liquid comprising water and ethanol having an ethanol content of >15% by weight based on the total weight of the liquid by means of the liquid inlet (21) and an inert gas by means of the gas inlet (22) and the nebulising means (2) is capable of nebulising the liquid and the inert gas to form an aerosol and thus to supply them into the mixing chamber (3),
   the mixing chamber (3) comprises an evaporation means (31) and a connection to the separation chamber (4) or consists thereof, wherein the mixing chamber (3) is arranged after the nebulising means (2) and connected thereto in such a way that the aerosol formed by means of the nebulising means (2) can be supplied into the mixing chamber (3) and the evaporation means (31) is capable of converting a part or the ethanol of the supplied aerosol partly or completely into the gas phase,
   the separation chamber (4) comprises a gas outlet (41) and a connection to the condensation chamber (5) or consists thereof, wherein the separation chamber (4) is arranged after the mixing chamber (3) and connected thereto in such a way that the ethanol converted into the gas phase and the remaining aerosol can be supplied into the separation chamber (4) and a part or the ethanol gas phase can be removed from the device (1) through the gas outlet (41) and
   the condensation chamber (5) comprises a liquid outlet (51) and a condensation means (52) or consists thereof, wherein the condensation chamber (5) is arranged after the separation chamber (4) and connected thereto in such a way that the aerosol remaining in the separation chamber (4) after partial or complete removal of the ethanol gas phase can be supplied into the condensation chamber (5) and the condensation means (5) is capable of wholly or partly condensing the supplied aerosol and the condensate having an ethanol content of ≦15% by weight based on the total weight of the condensate can be removed from the condensation chamber (5) through the liquid outlet (51).
Specific embodiment two comprises a device according to specific embodiment one, characterised in that the device (1), in the constructed state, is constructed perpendicularly to the centre of the earth and the nebulising means (2) are arranged at the upper end of the device (1) and the condensation chamber (5) at the lower end of the device (1).
Specific embodiment three comprises a device according to specific embodiment one or two, characterised in that the mixing chamber (3) additionally comprises a gas inlet (36) for supplying an inert gas into the mixing chamber (3).
Specific embodiment four comprises a device according to any one of the preceding specific embodiments, characterised in that the mixing chamber (3) is configured as a tube.
Specific embodiment five comprises a device according to any one of the preceding specific embodiments, characterised in that the separation chamber (4) comprises an equally large or larger cross section compared to the mixing chamber (3).
Specific embodiment six comprises a device according to any one of the preceding specific embodiments, characterised in that the gas outlet (41) is configured as a tube running through an outer wall (43) of the separation chamber (4) into a space outside the device (1), wherein there are arranged one end of the tube (411) inside the separation chamber (4) in a region opposing the connection to the mixing chamber (3) and a second end of the tube (412) in the space outside the device (1).
Specific embodiment seven comprises a device according to specific embodiment six, characterised in that the gas outlet (41) is arranged at the lower end of the separation chamber (5) in the constructed state and runs in a region above the lower end through the outer wall (43) of the separation chamber (4) into the space outside the device (1).
Specific embodiment eight comprises a device according to any one of the preceding specific embodiments, characterised in that the condensation chamber (5) is a tube.
Specific embodiment nine comprises a device according to any one of the preceding specific embodiments, characterised in that the liquid outlet (51) is arranged at the lower end of the device (1) in the constructed state.
Specific embodiment ten comprises a process for reducing an ethanol content in a flavouring substance preparation comprising or consisting of one or more flavouring substances, water and ethanol comprising or consisting of the following steps:
   a) providing a liquid flavouring substance preparation which comprises one or more flavouring substances, water and ethanol having an ethanol content of >15% by weight based on the total weight of the flavouring substance preparation or consists thereof, and also an inert gas,
   b) supplying the liquid flavouring substance preparation through a liquid inlet (21) and the inert gas through a gas entrance (22) into a nebulising means (2) by means of which the flavouring substance preparation and the inert gas are nebulised to form an aerosol,
   c) supplying the aerosol formed in step b) into a mixing chamber (3) which is connected to the nebulising means (2) and has evaporation means (31) and converting a part of or the entire ethanol component of the aerosol by means of the evaporation means (31) into a gas phase,
   d) forwarding the aerosol formed in step c) into a separation chamber (4) which is connected to the mixing chamber (3) and has a gas outlet (41) and removing a part of or the entire gaseous ethanol component from the separation chamber (4) through the gas outlet (41),
   e) forwarding the aerosol formed in step d) into a condensation chamber (5) which is connected to the separation chamber (4) and has a liquid outlet (51) and condensation means (52) and condensing the aerosol by means of the condensation means (52) to form a flavouring substance concentrate comprising or consisting of the flavouring substance or substances, water and ethanol having an ethanol content of ≦15% by weight based on the total weight of the flavouring substance concentrate and
   f) removing the flavouring substance concentrate formed in step e) from the condensation chamber (5) through the liquid outlet (51).
Specific embodiment ten comprises a process according to specific embodiment ten, characterised in that the flavouring substance preparation, which is supplied in step a) to the nebulising means (2), is an eluate obtained by means of a liquid chromatography system (6).
Specific embodiment twelve comprises a process according to specific embodiment ten or eleven, characterised in that the liquid chromatography system (6) is connected to the liquid inlet (21), so that the eluate is supplied immediately after separation in the liquid chromatography system (6) through the liquid inlet (21) into the nebulising means (2).
Specific embodiment thirteen comprises a process for sensory assessment of one or more flavouring substances comprising or consisting of the following steps:
   i. producing a liquid flavouring substance concentrate comprising or consisting of one or more flavouring substances, water and ethanol having an ethanol content of ≦15% by weight based on the total weight of the flavouring substance concentrate in accordance with one of the processes according to specific embodiments ten to eleven and
   ii. supplying the flavouring substance concentrate into the mouth of a test subject for sensory assessment.
Specific embodiment fourteen comprises the process according to specific embodiment thirteen, characterised in that the sensory assessment is carried out gustatorially and/or retronasally.
Specific embodiment fifteen comprises a use of a device (1) according to any one of specific embodiments one to nine for producing a liquid flavouring substance concentrate comprising or consisting of one or more flavouring substances, water and ethanol having an ethanol content of ≦15% by weight based on the total weight of the flavouring substance concentrate.

We claim:

1. A device for reducing an ethanol content in a liquid preparation comprising a nebulising means, a mixing chamber, a separation chamber and a condensation chamber, wherein
the nebulising means comprises a liquid inlet, a gas inlet and a connection to the mixing chamber or consists thereof and the nebulising means is operated at a temperature of ≦100° C., so that there can be supplied to the nebulising means a liquid comprising water and ethanol having an ethanol content of >15% by weight based on the total weight of the liquid by means of the liquid inlet and an inert gas by means of the gas inlet and the nebulising means is capable of nebulising the liquid and the inert gas to form an aerosol and thus to supply them into the mixing chamber,
the mixing chamber comprises an evaporation means and a connection to the separation chamber or consists thereof, wherein the mixing chamber is arranged after the nebulising means and connected thereto in such a way that the aerosol formed by means of the nebulising means can be supplied into the mixing chamber and the evaporation means is capable of converting a part or the ethanol of the supplied aerosol partly or completely into the gas phase,
the separation chamber comprises a gas outlet and a connection to the condensation chamber or consists thereof, wherein the separation chamber is arranged after the mixing chamber and connected thereto in such a way that the ethanol converted into the gas phase and the remaining aerosol can be supplied into the separation chamber and a part or the ethanol gas phase can be removed from the device through the gas outlet and
the condensation chamber comprises a liquid outlet and a condensation means or consists thereof, wherein the condensation chamber is arranged after the separation chamber and connected thereto in such a way that the aerosol remaining in the separation chamber after partial or complete removal of the ethanol gas phase can be supplied into the condensation chamber and the condensation means is capable of wholly or partly condensing the supplied aerosol and the condensate having an ethanol content of ≦15% by weight based on the total weight of the condensate can be removed from the condensation chamber through the liquid outlet.

2. The device according to claim 1, wherein the device, in the constructed state, is constructed perpendicularly to the centre of the earth and the nebulising means are arranged at the upper end of the device and the condensation chamber at the lower end of the device.

3. The device according to claim 1, wherein the mixing chamber additionally comprises a gas inlet for supplying an inert gas into the mixing chamber.

4. The device according to claim 1, wherein the mixing chamber is configured as a tube.

5. The device according to claim 1, wherein the separation chamber comprises an equally large or larger cross section compared to the mixing chamber.

6. The device according to claim 1, wherein the gas outlet is configured as a tube running through an outer wall of the separation chamber into a space outside the device, wherein there are arranged one end of the tube inside the separation chamber in a region opposing the connection to the mixing chamber and a second end of the tube in the space outside the device.

7. The device according to claim 6, wherein the gas outlet is arranged at the lower end of the separation chamber in the constructed state and runs in a region above the lower end through the outer wall of the separation chamber into the space outside the device.

8. The device according to claim 1, wherein the condensation chamber is a tube.

9. The device according to claim 1, wherein the liquid outlet is arranged at the lower end of the device in the constructed state.

10. A process for reducing an ethanol content in a flavouring substance preparation comprising or consisting of one or more flavouring substances, water and ethanol comprising or consisting of the following steps:
a) providing a liquid flavouring substance preparation which comprises one or more flavouring substances, water and ethanol having an ethanol content of >15% by weight based on the total weight of the flavouring substance preparation or consists thereof, and also an inert gas,
b) supplying the liquid flavouring substance preparation through a liquid inlet and the inert gas through a gas entrance into a nebulising means by means of which the flavouring substance preparation and the inert gas are nebulised to form an aerosol,
c) supplying the aerosol formed in step b) into a mixing chamber which is connected to the nebulising means and has evaporation means and converting a part of or the entire ethanol component of the aerosol by means of the evaporation means into a gas phase,
d) forwarding the aerosol formed in step c) into a separation chamber which is connected to the mixing chamber and has a gas outlet and removing a part of or the entire gaseous ethanol component from the separation chamber through the gas outlet,
e) forwarding the aerosol formed in step d) into a condensation chamber which is connected to the separation chamber and has a liquid outlet and condensation means and condensing the aerosol by means of the condensation means to form a flavouring substance concentrate comprising or consisting of the flavouring substance or substances, water and ethanol having an ethanol content of ≦15% by weight based on the total weight of the flavouring substance concentrate and
f) removing the flavouring substance concentrate formed in step e) from the condensation chamber through the liquid outlet.

11. The process according to claim 10, wherein the flavouring substance preparation, which is supplied in step a) to the nebulising means, is an eluate obtained by means of a liquid chromatography system.

12. The process according to claim 10, wherein the liquid chromatography system is connected to the liquid inlet, so that the eluate is supplied immediately after separation in the liquid chromatography system through the liquid inlet into the nebulising means.

13. A process for sensory assessment of one or more flavouring substances comprising or consisting of the following steps:
i. producing a liquid flavouring substance concentrate comprising or consisting of one or more flavouring substances, water and ethanol having an ethanol content of ≦15% by weight based on the total weight of the flavouring substance concentrate in accordance with one of the processes according to claim 10 and
ii. supplying the flavouring substance concentrate into the mouth of a test subject for sensory assessment.

14. A process according to claim 13, wherein the sensory assessment is carried out gustatorially and/or retronasally.

15. A method for producing a liquid flavouring substance concentrate comprising or consisting of one or more flavouring substances, water and ethanol having an ethanol content of ≦15% by weight based on the total weight of the flavouring substance concentrate, the method comprising reducing an ethanol content with a device according to claim 1.
